(19) United States
(12) Patent Application Publication
(10) Pub. No.: US 2026/0086917 A1
(43) Pub. Date: Mar. 26, 2026

(54) CODE GENERATION USING LLMS WITH FOREST SEARCH FOR MEDICAL DECISION MAKING

(71) Applicant: NEC Laboratories America, Inc., Princeton, NJ (US)

(72) Inventors: Wei Cheng, Princeton Junction, NJ (US); Xujiang Zhao, Hillsborough, NJ (US); Wenchao Yu, Plainsboro, NJ (US); Yiyou Sun, Madison, WI (US); Yanchi Liu, Princeton, NJ (US); Haifeng Chen, West Windsor, NJ (US); Jonathan Li, Troy, NY (US)

(21) Appl. No.: 19/329,835

(22) Filed: Sep. 16, 2025

Related U.S. Application Data
(60) Provisional application No. 63/699,321, filed on Sep. 26, 2024.

(51) Int. Cl. G06F 11/3604; G06F 8/35; G16H 40/63; G16H 50/20

(52) U.S. Cl. CPC G06F 11/3608; G06F 8/35; G16H 40/63; G16H 50/20

(57) ABSTRACT

Methods and systems for code generation include generating code for tree root nodes responsive to a query that specifies a task. The tree root nodes are expanded into trees based on foresting tree search using scattering to select varied directional prompts and sharing direction information between the trees. Generated code is output corresponding to a node from the trees that satisfies a test case.

### RELATED APPLICATION INFORMATION

This application claims priority to U.S. Patent Application No. 63/699,321, filed on Sep. 26, 2024, incorporated herein by reference in its entirety.

### BACKGROUND

### Technical Field

The present invention relates to large language models and, more particularly, to code generation.

### Description of the Related Art

Large language models (LLMs) can be trained to generate programming source code in any appropriate programming language. However, LLMs can generate outputs that appear to be valid at first glance, but which ultimately fail code tests and produce undesirable results.

### SUMMARY

A method for code generation includes generating code for tree root nodes responsive to a query that specifies a task. The tree root nodes are expanded into trees based on foresting tree search using scattering to select varied directional prompts and sharing direction information between the trees. Generated code is output corresponding to a node from the trees that satisfies a test case.

A system for code generation includes a hardware processor a memory that stores a computer program. When executed by the hardware processor, the computer program causes the hardware processor to generate code for a plurality of tree root nodes responsive to a query that specifies a task, to expand the plurality of tree root nodes into a plurality of trees based on foresting tree search using scattering to select varied directional prompts and sharing direction information between the plurality of trees, and to output generated code corresponding to a node from the plurality of trees that satisfies a test case.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a code generation system that uses scattered forest search to generate program code, in accordance with an embodiment of the present invention;

FIG. 2 is a block/diagram of a method for generating program code using scattered forest search, in accordance with an embodiment of the present invention;

FIG. 3 is a block/flow diagram of a method for performing a scattered forest search, in accordance with an embodiment of the present invention;

FIG. 4 is a block diagram of a healthcare facility that uses code generation with scattered forest search to assist in medical decision making;

FIG. 5 is a block diagram of a computing device that generates code using a scattered forest search, in accordance with an embodiment of the present invention;

FIG. 6 is a diagram of an exemplary neural network model that can be used to implement part of a low-level coder with forest search, in accordance with an embodiment of the present invention; and

FIG. 7 is a diagram of an exemplary deep neural network model that can be used to implement part of a low-level coder with forest search, in accordance with an embodiment of the present invention.

### DETAILED DESCRIPTION OF PREFERRED EMBODIMENTS

While large language models (LLMs) can be effective in generating programming source code, they have limitations. For example, a single LLM may struggle to fully address a complex or multi-part query, particularly when they involve deep domain knowledge or integration of multiple programming concepts. In contrast, different LLMs can be trained to specialize in particular programming languages or frameworks, making the overall system more scalable and adaptable to the specific needs of a particular problem. In a multi-agent system, one LLM can generate code while others review or debug it, leading to higher accuracy and a better pass rate in code assessments. The multiple LLMs can handle ambiguities in user queries more effectively using their varied training and perspectives to interpret and clarify uncertain aspects before generating a code output.

The present embodiments improve the efficiency and accuracy of code generation using LLMs, particularly in complex, multi-component, or highly specialized programming tasks. To that end, a non-parametric approach is used that fosters self-improvement with minimal human involvement. Multiple LLMs collaborate, coupled with a forest search using a Monte Carlo tree search (MCTS) to simulate and validate different code outputs. This provides code quality feedback so that new code can be generated that addresses the feedback.

A multi-tree search, or forest search, helps to avoid local optima using both a local idea list and a global idea list to introduce diversity in query prompts. An ant colony approach is used to optimize idea evolution, to help find ideas that the LLM agents can use to generate code.

Referring now to FIG. 1, a code generation system is shown. A high-level planner 102 receives a prompt 104 that specifies, for example, the coding task that is to be performed and, optionally, a resource budget for the LLM. The high-level planner 102 breaks the coding task into discrete parts that the low-level coder 106 can better handle, for example identifying a list of discrete sub-tasks and prompting the low-level coder 106 with them individually.

The low-level coder 106 uses an LLM to generate code and uses a forest search to guide the code generation, which it passes to code reviewer 108. The code reviewer 108 generates execution feedback of test cases and gives scores for each code node in the forest to judge which nodes to expand on. The code reviewer 108 provides the feedback to the low-level coder 106 and provides instructions about how to further explore the code generation space back to the high-level planner 102.

The high-level planner 102 makes use of global strategies to guide the low-level coder 106. The global strategy may be refined during iterations. A list of ideas is further maintained to improve the nodes in the code generation tree based on the latest feedback from the code reviewer 18. The global strategy and the list of ideas are included in the prompt that the high-level planner 102 sends to the low-level coder 106 for a next round of code generation. The global strategy and the list of ideas help to avoid local optima, preventing the output 110 from being locked into an incorrect state.

To this end, multiple trees may be searched, with different initial roots to start different MCTS attempts. The search will be refined to focus on promising trees, with good starting points of code to further refine until all tests are satisfied. A scattered forest search strategy may be used, where scattering dynamically varies input prompts when sampling from LLMs, driving more diverse and exploratory outputs. In scattering, the LLM suggests different textual optimization directions and steps, analogous to gradients in numerical optimization, before advancing toward a new solution. During tree search refinement, scattering effectively perturbs or mutates the previous solutions, resulting in an evolutionary search process.

Foresting is performed as the tree search equivalent of multi-start optimization, where scattering helps to diversify initialization seeds, ensuring that they are well-distributed through the search space. This enhances the breadth of exploration while effectively mitigating clearly incorrect solutions, such as those with syntax errors.

Scouting is used to enhance the scattering by sharing feedback and experience across search branches. When one branch discovers positive or negative results by following a specific textual direction, this information is relayed to guide future search steps, encouraging or discouraging exploration in that direction. Scouting thereby improves exploitation by intensifying the search around promising textual directions.

Stated formally, in a program synthesis task x= [symbol] p, H [symbol] , a solver is given the prompt p in natural language, asking the solver to write code for some object s. The goal is to complete the code implementation of s such that it passes all hidden tests H. The solver cannot see the hidden tests. The solver may, in some cases, be given validation tests V that it can use to test the solution s before submitting it for evaluation on the hidden tests H. The solver can also generate its own validation tests. Both the hidden tests and the validation tests may be in the form of a set of assert statements, which test the functionalities of s.

A solution s′ is considered to be correct if it passes all the hidden tests H. The solver is allowed k submissions [s]ₖ to the hidden tests. If at least one submission s* passes all the hidden tests, then the task is considered to be solved. Given a set of tasks X, the proportion of tasks [symbol] p, H [symbol] ∈X that are solved by the agent is called the pass@k rate.

Three optimizations are used to enhance both exploration and exploitation of tree search using LLMs. In a tree search, child solutions of a given parent solution tend to be similar to one another, since the LLM is given the same prompt to generate the children. Exploration is encouraged when generating child solutions by querying the LLM to generate possible improvement directions [d]ₙ first. The LLM is then instructed to implement a specific direction dⱼ for each child sᵢⱼ that it generates from parent sᵢ. This helps to explore different, often orthogonal, improvement directions to cover a wider region of the search space. This effectively scatters the tree's branches, encouraging the tree search to explore more diverse solutions using varied directional prompts.

The selection of directions for exploration may be implemented using an upper confidence tree (UCT) approach in MCTS:

$$UCT ( s , d ) = \hat{Q} ( s , d ) + c \sqrt{\frac{ln ( {\sum }_{b} n ( s , b ) )}{n ( s , d )}}$$

where c is an exploration parameter, n(s,d) is the number of visits of direction d at solution s, and \hat{Q} (s,d) is the estimated q-value, which is updated via backpropagation as follows:

$${\hat{Q} ( {s}_{i} , {d}_{i + 1} )}^{t + 1} ← ( 1 - {α}_{n} {\hat{Q} ( {s}_{i} , {d}_{i + 1} )}^{( t )} + {α}_{n} max \{ {\hat{Q} ( {s}_{i} , {d}_{i + 1} )}^{( t )} , {\hat{Q} ( {s}_{i + 1} , {d}_{i + 2} )}^{( t + 1 )} \} )$$

where αₙ is the weighted average parameter that depends on n(s,d). The backpropagation occurs along the entire MCTS simulated trajectory τ^{(t+1)}=[s₀, d₁, s₂, . . . , s₋₂, d₋₁, s₋₁], where the q-value for the penultimate state s₋₁ is updated using the value of the final state (e.g., a percentage of validation states passed): \hat{Q}(s₋₂, d₋₁)^{(t+1)}←v(s₋₁), where v(s) is a value of the state s. The final state value comes from the reward model.

The term selecting the maximum of \hat{Q}(sᵢ, dᵢ₊₁)^{(t)} and \hat{Q}(sᵢ₊₁, dᵢ₊₂)^{(t+1)} ensures that, if the next solution is worse, the current solution can be used instead. This approach dynamically selects which direction to explore, prioritizing more promising parent solutions sⱼ over exploring all directions of a parent solution sᵢ. Using UCT to select distinct actions is effective in balancing exploration and exploitation.

Iterative refinement faces a challenge in that a very faulty initial seed solution may be difficult to effectively correct during the search process, for example if it starts near a local optimum. Multiple seed solutions may be used, with the tree search being performed from each one. This is referred to herein as foresting, where n seed solutions [s]ₙ are generated and the seed function is dynamically selected to evolve from UCT. Specifically, for each MCTS simulation, seed function sᵢ is selected with the highest UCT value and the simulation is conducted from that point. Similar to branch scattering, more diverse seed solution generation is promoted through forest scattering by providing the LLM with different instructions prior to generating solutions.

Solutions may furthermore be improved using insights from effective improvement directions. After generating a new solution s₋₁ using improvement directions d₋₁ on s₋₂, feedback f₋₁ is given to the LLM to assess the effectiveness of the direction and to derive general insights. These insights are stored in a list and included in future prompts, enabling the sharing of knowledge of effective improvements across branches. This enhances feedback exploitation and strengthens the scattering.

The search strategy may be defined as a Markov transition kernel P(s, s′), which denotes the probability of generating a new solution s′ given the current solution s. A chain of self-refined solutions s₀, s₁, . . . is generated following the transition kernel P. The transition kernel may be expressed as:

$${P}_{previous} ( s , {s}^{′} ) = {π}_{c} ( {s}^{′} \vert s , F ( s ) )$$

where π_{c} an LLM with a concentrated policy that is conditioned on the previous solution s and the feedback F(s). The LLM's output can be highly concentrated and may output highly similar solutions.

With scattering, an improvement direction d generated by the LLM is sampled with prompt π(⋅|s,F(s)). The LLM π is then prompted to generate the next solution s′ given the current solution s, the feedback, and the improvement direction d. The scattering transition kernel may be expressed as:

$${P}_{scattering} ( s , {s}^{′} ) = {\sum }_{d} {π}_{t} ( d \vert s , F ( s ) ) {π}_{c} ( {s}^{′} \vert s , F ( s ) , d )$$

where πₜ is a text-based reflection policy that can generate highly diverse improvement directions.

A diverse transition kernel increases the probability of moving between different regions of the state space [symbol] , enhancing the conductance Φ of the chain:

$${Φ}_{P} ( S ) = \frac{{\sum }_{s ∈ S , {s}^{′} ∉ S} μ ( s ) P ( s , {s}^{′} )}{μ ( S )}$$

where S is a subset of state space S and μ is a stationary distribution. Higher conductance improves the spectral gap γ, which is inversely related to the mixing time of the Markov chain, thereby reducing the likelihood of the chain getting trapped in local regions. For a local region S, a scattering search can generate highly diverse directions d and lead to new solutions s′ out of the local region, where P_{scattering}(s, s′)>0 if some direction d gives the correct direction.

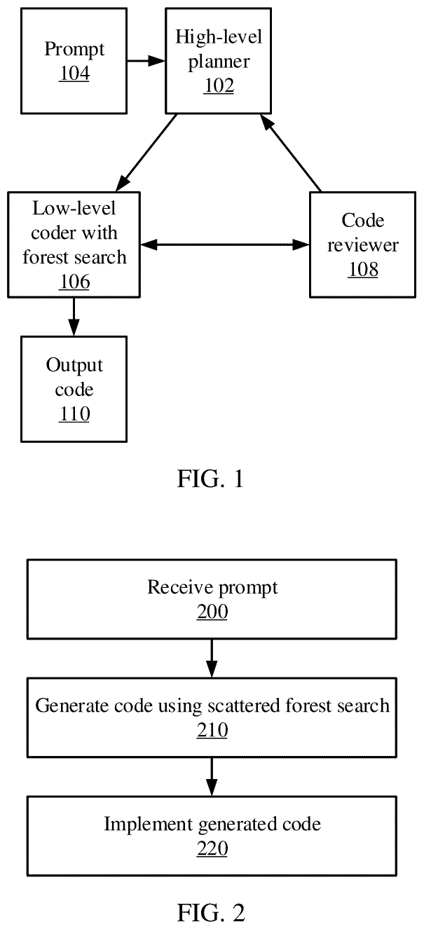

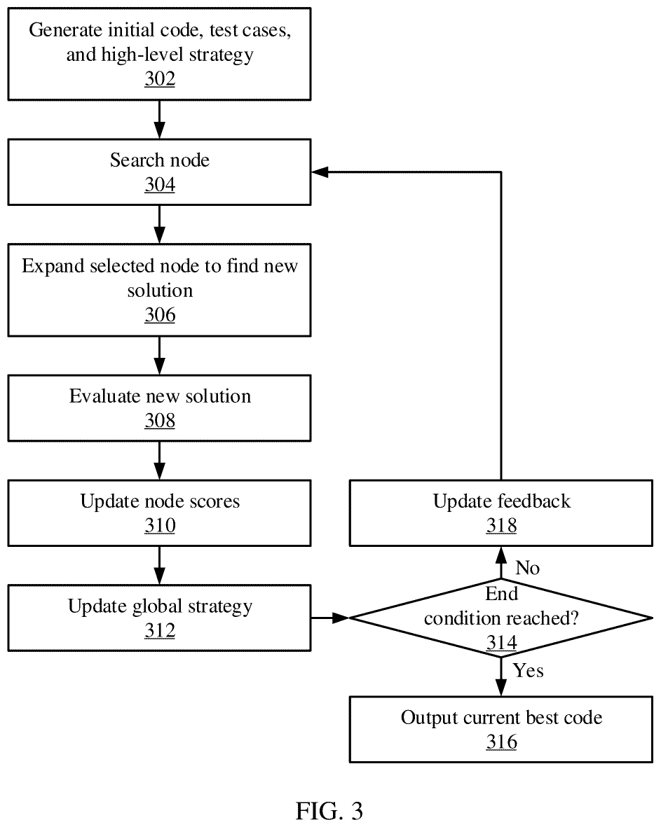

Referring now to FIG. 2, a method of generating code is shown. Block 200 receives a prompt that specifies initial code requirements, possible test cases, and an LLM query budget. This query may be expressed using natural language with any appropriate level of specificity, for example defining the intended function of the program code that is to be generated.

Block 210 generates code using a scattered forest search with scouting, as described above and as shown in greater detail below. The code generation of block 210 seeks to find optimal solutions by performing tree searches from multiple diverse starting positions, using a scattering approach and scouting to explore the solution space and to share information between individual tree searches.

Block 220 then implements the generated code. The code generated in block 210 pasts validation tests and hidden tests and so may be used to execute its intended function. In embodiments where the generated code is in an interpreted language, it may be executed directly. In embodiments where the generated code is in a compiled language, the generated source code may first be compiled to an executable form. In some cases block 220 may integrate the generated code with a code repository, where it performs functions within a larger program.

It is particularly contemplated that the code may be implemented in the medical domain, where clinician-in-the-loop processes may use the generated code for informed decision making. In an example, multiple candidate treatment plans for a patient may be generated and selected for those which pass tests relating to guideline adherence, contraindications, and patient-specific constraints. The code generation may further be used for diagnostic workup sequencing, where diversified next-test options are explored, and branches are selected that satisfy sensitivity and specificity thresholds, cost, radiation limits, and pre-test probability updates. The code generation may further be used to select medication and dosing for a patient, with alternative regimens and dosages being proposed and only those solutions which pass checks for dosing rules, drug interactions, and allergy exclusions being selected.

Referring now to FIG. 3, additional detail on the generation of code in block 210 is shown. Block 302 generates initial code responsive to the input code requirements, with multiple different initial code samples being generated to perform as the root notes of the forest in the search space, together with test cases and a global strategy to solve the problem. Block 304 then searches one of the nodes in the forest. The initial node or nodes may be searched randomly, and subsequent nodes may be selected based on their scores to further expand into the search space, making use of any available feedback from test cases to make improvements.

Block 306 expands the selected node to a new solution, for example using scattering, to identify a direction that is added to the list of improvement ideas. These improvement ideas may be added to the LLM's prompt to improve subsequent code solutions. Block 308 uses the test case(s) to evaluate the new solution, calculating a corresponding score. The scores of the parent nodes may be updated by block 310, with recursive updates to parent nodes back to the root of the tree. A leaf node's score is calculated using a reward model. For coding, an LLM can generate a set of test cases given the coding problem, and these test cases can be used on the generated code. The percentage of passed test cases can be used as the leaf node's score. The score is then back-propagated to the parent nodes using UCT.

Block 312 updates the global strategy based on a comparison of the score of the newly generated node to the current best, using the current list of ideas. If the newly generated node is not an improvement over the current best, then block 312 may be skipped.

Block 314 determines whether an end condition has been reached, for example if the query budget limit has been reached or if all test cases have been passed. If so, block 316 outputs the current best solution as a response to the query. If not, block 318 updates feedback for the selection of a next node and processing returns to block 304.

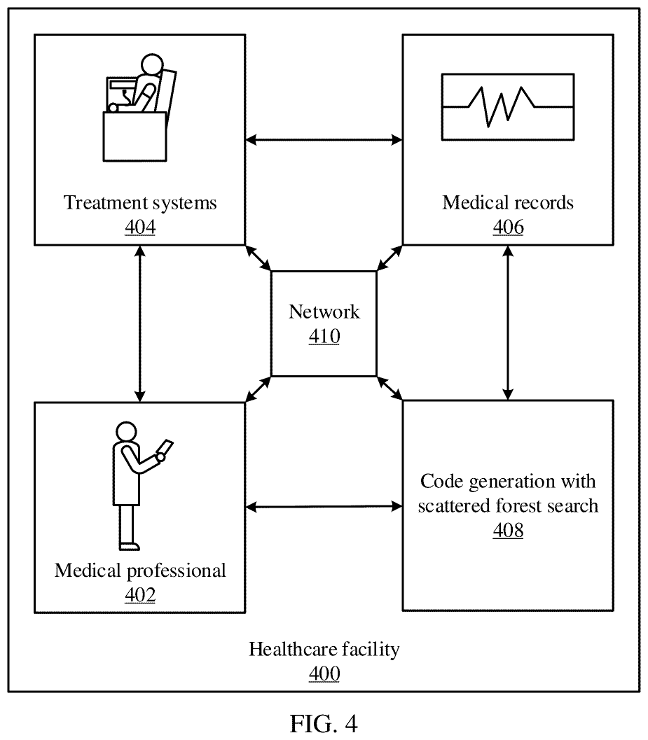

Referring now to FIG. 4, a diagram of time series analysis is shown in the context of a healthcare facility 400. Code generation with scattered forest search 408 may be used to aid in medical decision making, for example by generating program code responsive to a patient's particular condition. In a medical context, the code may be generated to control treatment systems 404, for example including instructions as to timing and type of treatments. In some cases the generated code may be used to assess a patient's health condition, referring to the patient's medical records 406, and may make recommendations or take actions to treat the patient.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Code generation with scattered forest search 408 may be prompted with a task to generate specialized control code based on a patient's specific medical condition, indicated by the medical records 406, and input from medical professionals 402. This may include test cases that are based in the patient's particular sensitivities, for example indicating conditions that would be hazardous to the patient's health.

The different elements of the healthcare facility 400 may communicate with one another via a network 410, for example using any appropriate wired or wireless communications protocol and medium. Thus the code generation with scattered forest search 408 receives data from treatment systems 404, medical professionals 402, and from medical records 406, and generates control code for a treatment that is safe for the patient. The generated control code may further coordinate with treatment systems 404 in some cases to automatically administer or alter a treatment. For example, the control code may automatically time and dose a treatment to achieve a therapeutic effect without overwhelming the patient.

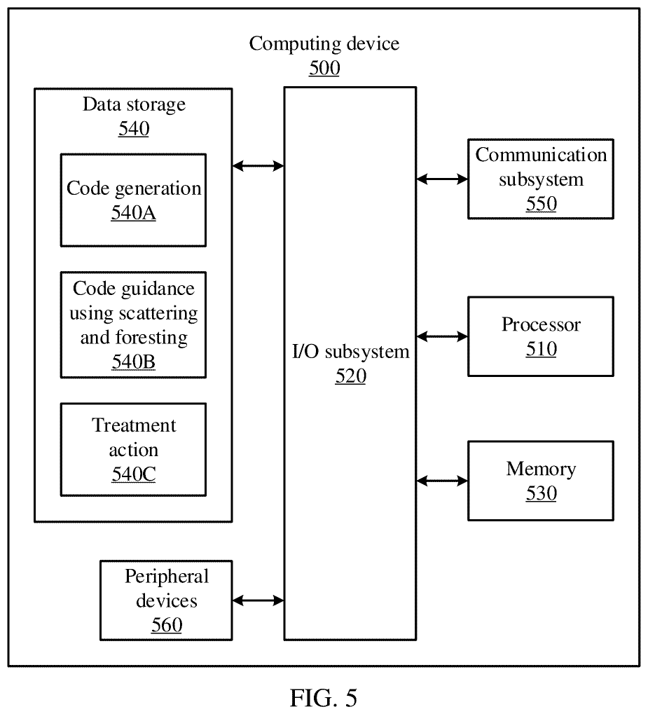

Referring now to FIG. 5, an exemplary computing device 500 is shown, in accordance with an embodiment of the present invention. The computing device 500 is configured to perform code generation.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for code generation, 540B for guiding the code generation using scattering and foresting strategies, and/or 540C for performing treatment actions using generated code. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

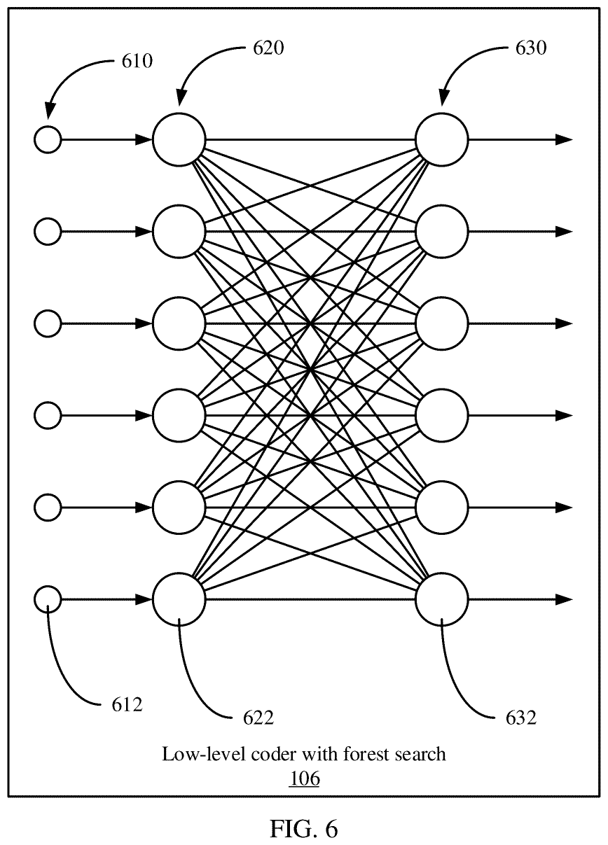

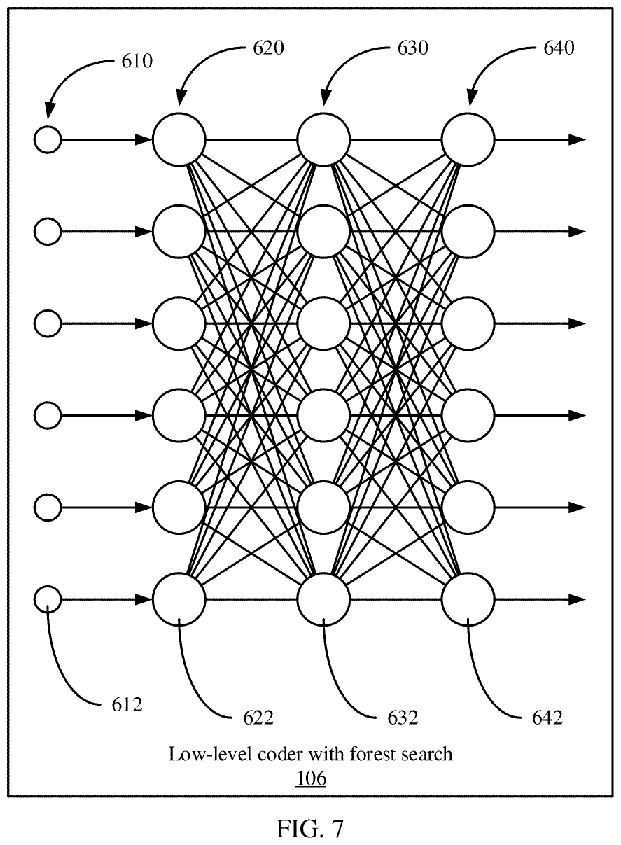

Referring now to FIGS. 6 and 7, exemplary neural network architectures are shown, which may be used to implement parts of the present machine learning models, such as the low-level coder with forest search 106. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for code generation, comprising:
generating code for a plurality of tree root nodes responsive to a query that specifies a task;
expanding the plurality of tree root nodes into a plurality of trees based on foresting tree search using scattering to select varied directional prompts and sharing direction information between the plurality of trees; and
outputting generated code corresponding to a node from the plurality of trees that satisfies a test case.

2. The method of claim 1, wherein scattering dynamically selects directions for the foresting tree search using an upper confidence tree.

3. The method of claim 1, wherein expanding the plurality of tree root nodes includes selecting a node from one of the plurality of trees and generating new code for a new node in the one of the plurality of trees, based on a selected direction in a search space.

4. The method of claim 1, wherein iterations of the foresting tree search selects between the plurality of trees with a highest upper confidence tree value.

5. The method of claim 1, wherein sharing directional information between the plurality of trees includes adding directions from previous iterations of expanding the plurality of tree root nodes into a plurality of trees into a prompt for code generation.

6. The method of claim 1, wherein generating code and expanding the plurality of tree root nodes are implemented using a machine learning model that includes a large language model.

7. The method of claim 6, wherein expanding the plurality of root nodes includes iteratively prompting the large language model with feedback based on performance of previous nodes in the plurality of trees under test cases.

8. The method of claim 1, wherein expanding the plurality of tree root nodes includes determining a performance score for each newly generated code and updating scores of parent nodes in a respective tree of the plurality of trees.

9. The method of claim 1, wherein the task is based in medical decision making for a patient and wherein the output generated code implements control of a treatment system.

10. The method of claim 9, further comprising executing the output generated code to control the treatment system and to automatically perform treatment of the patient.

11. A system for code generation, comprising:
a hardware processor; and
a memory that stores a computer program which, when executed by the hardware processor, causes the hardware processor to:
generate code for a plurality of tree root nodes responsive to a query that specifies a task;
expand the plurality of tree root nodes into a plurality of trees based on foresting tree search using scattering to select varied directional prompts and sharing direction information between the plurality of trees; and
output generated code corresponding to a node from the plurality of trees that satisfies a test case.

12. The system of claim 11, wherein the scattering dynamically selects directions for the foresting tree search using an upper confidence tree.

13. The system of claim 11, wherein the expansion of the plurality of tree root nodes includes selection of a node from one of the plurality of trees and generation of new code for a new node in the one of the plurality of trees, based on a selected direction in a search space.

14. The system of claim 11, wherein iterations of the foresting tree search selects between the plurality of trees with a highest upper confidence tree value.

15. The system of claim 11, wherein the sharing of directional information between the plurality of trees includes adding directions from previous iterations of expanding the plurality of tree root nodes into a plurality of trees into a prompt for code generation.

16. The system of claim 11, wherein generation of code and expansion of the plurality of tree root nodes are implemented using a machine learning model that includes a large language model.

17. The system of claim 16, wherein expansion of the plurality of root nodes includes iteratively prompting the large language model with feedback based on performance of previous nodes in the plurality of trees under test cases.

18. The system of claim 11, wherein expansion of the plurality of tree root nodes includes determination of a performance score for each newly generated code and an update of scores of parent nodes in a respective tree of the plurality of trees.

19. The system of claim 11, wherein the task is based in medical decision making for a patient and wherein the output generated code implements control of a treatment system.

20. The system of claim 19, wherein the computer program further causes the hardware processor to execute the output generated code to control the treatment system and to automatically perform treatment of the patient.
